(19) United States
(12) Patent Application Publication
(10) Pub. No.: US 2017/0088849 A1
(43) Pub. Date: Mar. 30, 2017

(54) USE OF TANAC2 PROTEIN AND ENCODING GENE THEREOF

(71) Applicant: INSTITUTE OF GENETICS AND DEVELOPMENTAL BIOLOGY CHINESE ACADEMY OF SCIENCES, Beijing (CN)

(72) Inventors: Yiping TONG, Beijing (CN); Xue HE, Beijing (CN); Xueqiang ZHAO, Beijing (CN); Baoyuan QU, Beijing (CN); Wenying MA, Beijing (CN); Wenjing LI, Beijing (CN); Xia HONG, Beijing (CN); Ji SHI, Beijing (CN); Bin LI, Beijing (CN); Zhensheng LI, Beijing (CN)

(21) Appl. No.: 14/916,168

(22) PCT Filed: Feb. 13, 2015
(86) PCT No.: PCT/CN2015/000094

§ 371 Date: Mar. 2, 2016

(30) Foreign Application Priority Data
May 26, 2014 (CN) 201410224957.4

(51) Int. Cl. C12N 15/82; C07K 14/415

(52) U.S. Cl. CPC C12N 15/8261; C07K 14/415

(57) ABSTRACT

The TaNAC2 gene can promote uptake and utilization of nitrogen in a plant. A method for improving the uptake, transport and/or assimilation of nitrogen element in the plant includes: introducing a gene encoding a protein represented by SEQ ID NO:4 into a primary plant to obtain a transgenic plant. Compared to the primary plant, the uptake, transport and/or assimilation of the nitrogen element in the transgenic plant are improved. The method provides advantages in that: the TaNAC2 gene, as a nitrate nitrogen responsive regulatory factor, which can regulate the expression of a series of genes in nitrogen element uptake pathways of wheat, greatly promotes the study of the metabolism and utilization of the nitrogen element in a plant; and it is possible to improve the assimilation efficiency of nitrogen element by increasing the expression of TaNAC2, thereby reducing fertilizer application and increasing yield.

### TECHNICAL FIELD

The present invention belongs to biological technology, and relates to a transcription regulating factor, a coding gene thereof, and use thereof; particularly to a TaNAC2 protein, a coding gene thereof, and use thereof in regulating uptake and use of nitrogen element in a plant.

### BACKGROUND ART

Wheat is one of important crops in China, and there is the world-wide greatest production thereof in China in terms of whether cultivation area or total production. Wheat was produced with a low per unit area yield in the early years of the People's Republic of China, e.g., 0.73 ton/hectare (48.8 kg/mu) in 1952, but its yield reached 4.55 ton/hectare (303.3 kg/mu) by 2006, an increase of 5.2 folds. One of the important reasons is an increased use of fertilizers. It was reported that fertilizers comprised 32% of production increase, 25% of production costs, and 50% of total production material expenses in agriculture. At present, the annual consumption of nitrogen fertilizers exceeds 24 Mt (pure nitrogen) in China, comprising above 30% of the annual consumption in the world (Ju et al., 2004). However, there is a lower utilization of the nitrogen fertilizers of only 30-35% in China (which is 45% in developed countries), and as much as 45-50% of the nitrogen fertilizers are not absorbed by crops, but lost into the environment, resulting in resource waste and environmental pollution (Zhu and Chen, 2002). As arable area is continuously reduced, increased yields of crops such as wheat still rely on increasing the application of fertilizers to satisfy an increasing demand of food. Therefore, improving the efficiency in nitrogen element uptake of wheat with biological means is of a strategic significance in ensuring food security and sustainable agricultural development.

The uptake of nitrogen in a plant comprises two stages: uptake, transport of nitrogen element, and assimilation of nitrogen element, and the genes involved therein was best studied in a plant model of Arabidopsis thaliana. The uptake of a plant to nitrate depends on two nitrate transport systems: a high affinity nitrate transporter, and a low affinity nitrate transporter. The high affinity transporter functions when the concentration of nitrate is lower; and AtNRT2.1 and AtNRT2 were first cloned as high affinity nitrate transporter genes (Filleur and Daniel-Vedele, 1999; Zhuo et al., 1999), and then another five genes from the same family were found through retrieval based on homology (Initiative, 2000). The low affinity transporter functions when the concentration of nitrate is higher; and AtNRT1.1 (CHL1) was cloned from an anti-chlorate (nitrate analog) mutant (Tsay et al., 1993). After entering into a plant body, nitrate is reduced by nitrate reductase (NR) and nitrite reductase (NiR) to ammonium nitrogen (NH4⁺), and get into amino acid metabolic pathways by means of glutamine synthetase (GS) and glutamate dehydrogenase (GDH). Studies shows that over-expression of GS, GOGAT and GDH can significantly improve the assimilation efficiency of nitrogen element and the production of crops (Ameziane et al. 2000; Habash et al. 2001; Miflin and Habash 2002).

Above studies merely stay at the level of a single functional gene. However, uptake and assimilation of nitrogen and phosphorus are actually regulated in a crossed and network-like way, involving processes such as photosynthesis, energy metabolism, etc., and it is usually difficult to improve the utilization of nutrients by a plant through over-expressing a gene of a pathway. Dof is a special set of transcription factors in plants. Recent studies indicate that over-expression of Dof1 gene in Arabidopsis can significantly improve (˜30%) net nitrogen content in and nitrogen deficiency resistance of Arabidopsis (Yanagisawa et al., 2004), based on the principle that Dof1 can upregulate the expressions of key genes encoding phosphoenolpyruvate carboxylase (PEPC), pyruvate kinase (PK), etc. in carbon element assimilation, to improve carbon metabolic pathways and accumulate carbon skeletons required for synthesis of amino acids, thereby increasing protein contents. Thus, finding a new regulatory factor regulating nitrogen and phosphorus responses of a plant can not only facilitate understanding the gene regulation network of a plant adapting to nitrogen and phosphorus stresses, but also provide a new gene source for cultivation of a new nutrient effective crop.

NAC transcription factors are special regulatory transcription factors in plants. Since the first NAC transcription factor in a plant was cloned from Petunia hybrida in 1996, NAC transcription factors have been found in Arabidopsis (Arabidopsis thaliana), rice, wheat, soybean (Glycine max) and other species so far. Arabidopsis contains at least 107 NAC genes (Riechmann et al., 2000), and rice contains 140 NAC genes (Fang et al., 2008). For the NAC transcription factors, their constitutions are primarily characterized by a highly conservative NAC domain at N-terminus of each of members. The NAC domain comprises about 160 amino acid residues, may be divided into 5 subdomains of I, II, III, IV, and V (Ooka et al., 2003), and possibly is responsible for binding with DNA and other proteins (Ernst et al., 2004). NAC protein has a less conservative C-terminus, having a transcription activating function (Ren et al., 2000; Xie et al., 2000; Duval et al., 2002). These studies show that, NAC proteins are involved in multiple growth and development process such as seed germination (Kim et al., 2008), secondary cell wall synthesis (Kubo et al., 2005; Ko et al., 2007), organ boundary and meristem formation (Aida et al., 1997; Takada et al., 2001; Vroemen et al., 2003; Mao et al., 2007), flowering (Kim et al., 2007), senescence (Guo and Gan, 2006; Yoon et al., 2008; Uauy et al., 2006) of a plant. NAC proteins also play an important role in a plant during biotic stresses, e.g. diseases (Collinge and Boller, 2001; Jensen et al., 2007; Bu et al., 2008). To date, there are many NAC proteins found being involved in the response of a plant to a stress. For example, SNAC1/2 (stress-responsive NAC 2) gene from rice has its expression inducible by drought, high salt, low temperature, injury and ABA, and when it is over-expressed, the plant has resistances to cold, salt, drought and the like (Hu et al., 2008); over-expression of wheat TaNAC2 in Arabidopsis also allows Arabidopsis to improve the resistance to non-biotic stresses (Mao et al., 2012). Among NAC proteins, a TaNAC2 gene from “Chinese Spring” wheat has a nucleotide sequence represented by 7^{th} to 993^{rd} nucleotide from 5′ end of SEQ ID No.3, and a TaNAC2 protein has an amino acid sequence represented by SEQ ID No.4. In summary, the NAC proteins, as a group of essential transcription factors, play an important role in regulation of growth and development and in various stress defending responses of plants, however, the study on NAC proteins in terms of improving nutrient uptake and particularly regulating nitrate uptake rate in plants is rarely reported.

### Invention Disclosure

An object of the present invention is to provide applications of a TaNAC2 protein and an encoding gene thereof which can promote uptake and utilization of nitrogen in a plant.

The present invention provides a method for improving uptake, transport and/or assimilation efficiencies of nitrogen element in a plant, comprising steps of: introducing a coding gene of a protein represented by SEQ ID No.4 into a primary plant to obtain a transgenic plant, which, compared with the primary plant, has improved uptake, transport and/or assimilation efficiencies of nitrogen element.

In above method, the coding gene may be introduced via a recombinant vector. The recombinant vector may be obtained by replacing the sequence of GUS gene of a modified pACH25 with the coding gene, particularly by inserting a DNA molecule represented by SEQ ID No.3 into the modified pACH25 between BamHI and KpnI enzyme cleavage sites, to replace the sequence of GUS gene therein.

The modified pACH25 may be obtained by inserting an Ubiquitin promoter into a pACH25 vector between PstI enzyme cleavage sites.

The introduction of the coding gene of the protein represented by SEQ ID No.4 into the primary plant may be performed by a transformation method mediated by a gene gun;

The nucleotide sequence of the coding gene may be particularly represented by 7th to 993rd nucleotide from 5′ end of SEQ ID No.3.

In any of above methods, the plant may be wheat.

A use of any of:
(1) a protein represented by SEQ ID No.4;
(2) a coding gene of the protein represented by SEQ ID No.4; and
(3) a recombinant vector, an expression cassette, a transgenic cell line, or a recombinant strain containing the coding gene in (2),

for improving nitrogen element uptake, transport and/or assimilation efficiencies of a plant, is also within the protection scope of the present invention.

A use of any of:
(1) a protein represented by SEQ ID No.4;
(2) a coding gene of the protein represented by SEQ ID No.4; and
(3) a recombinant vector, an expression cassette, a transgenic cell line, or a recombinant strain containing the coding gene in (2),

for promoting growth, development and/or grain production of a plant, is also within the protection scope of the present invention.

A use of any of:
(1) a protein represented by SEQ ID No.4;
(2) A coding gene of the protein represented by SEQ ID No.4; and
(3) a recombinant vector, an expression cassette, a transgenic cell line, or a recombinant strain containing the coding gene in (2),

for promoting root growth of a plant, is also within the protection scope of the present invention.

A use of any of:
(1) a protein represented by SEQ ID No.4;
(2) A coding gene of the protein represented by SEQ ID No.4; and
(3) a recombinant vector, an expression cassette, a transgenic cell line, or a recombinant strain containing the coding gene in (2),

for promoting increases of main root length and/or lateral root number, is also within the protection scope of the present invention.

A use of any of:
(1) a protein represented by SEQ ID No.4;
(2) A coding gene of the protein represented by SEQ ID No.4; and
(3) a recombinant vector, an expression cassette, a transgenic cell line, or a recombinant strain containing the coding gene in (2),

for improving the nitrate uptake rate of a plant, is also within the protection scope of the present invention.

A use of any of
(1) a protein represented by SEQ ID No.4;
(2) A coding gene of the protein represented by SEQ ID No.4; and
(3) a recombinant vector, an expression cassette, a transgenic cell line, or a recombinant strain containing the coding gene in (2),

for improving the tiller number or aboveground dry weight of a plant, or for improving the grain yield per plant, or for improving the nitrogen content in the body of a plant, or for improving the nitrogen concentration in the grains of a plant, is also within the protection scope of the present invention.

In any of above uses, the plant may be wheat.

The nucleotide sequence of the coding gene may be represented by 7th to 993rd nucleotide from 5′ end of SEQ ID No.3.

The present invention is advantageous in that:

1. although some key genes in processes such as nitrogen element uptake, assimilation, transport and the like have been cloned so far, since uptake and utilization of nitrogen element in a plant is regulated in crossed and network-like way, single functional gene is not sufficient to illuminate its regulation network; and the TaNAC2 gene cloned in the present invention, as a nitrate nitrogen responsive regulatory factor, allows for regulating the expression of a series of genes in the pathway of uptake and utilization of nitrogen element in wheat, which greatly advances the mechanism study for illuminating the metabolic utilization of the nitrogen element in a plant;

2. the TaNAC2 gene can improve nitrate uptake rate, grain yield per plant, total plant nitrogen content, grain nitrogen concentration, and nitrogen element harvest index of wheat, indicating that the increase of the expression of TaNAC2 through gene engineering enables the improvement of nitrogen element assimilation efficiency, thereby realizing the purposes of reducing fertilizer application and increasing yield and the like;

3. the TaNAC2 gene does not only have an effect on nitrogen element uptake, but also increases the number of lateral roots and the length of main root of wheat when over-expressed, so the study of the function of the gene is significant for the research of effective utilization of water and fertilizer by plants.

### BRIEF DESCRIPTION OF DRAWINGS

FIG. 1 is a schematic diagram of a pACH25/TaNAC2 vector.

FIG. 2 shows identification of a transgenic plant at DNA level.

FIG. 3 shows identification of a transgenic plant at RNA level.

FIG. 4 shows root appearances of transgenic and wild-type plants in high- and low-nitrogen culture conditions.

FIG. 5 shows nitrate uptake rates of roots of transgenic and wild-type plants in high- and low-nitrogen culture conditions.

FIG. 6 shows tiller numbers of transgenic and wild-type plants in high- and low-nitrogen culture conditions.

FIG. 7 shows aboveground dry weights of transgenic and wild-type plants at seeding stage in high- and low-nitrogen culture conditions.

FIG. 8 shows grain yields per plant of transgenic and wild-type plants in high- and low-nitrogen culture conditions.

FIG. 9 shows total nitrogen content determination of transgenic and wild-type plants at seeding stage in high- and low-nitrogen culture conditions.

FIG. 10 shows grain nitrogen concentrations of transgenic and wild-type plants in high- and low-nitrogen culture conditions.

FIG. 11 shows nitrogen element harvest indices of transgenic and wild-type plants in high- and low-nitrogen culture conditions.

### BEST MODE TO CARRY OUT THE INVENTION

The “Chinese Spring” wheat (Triticum aestivum L.) is disclosed in Brenchley R, Spannagl M, Pfeifer M, et al. Analysis of the bread wheat genome using whole-genome shotgun sequencing[J]. Nature, 2012, 491(7426): 705-710, and is publicly available from the Institute of Genetics and Developmental Biology, Chinese Academy of Sciences.

The KOD plus DNA polymerase is purchased from the TOYOBO Co.

The 10×PCR buffer for KOD plus is purchased from the TOYOBO Co.

The pACH25 vector is disclosed in Christensen A H, Quail P H, 1996, Ubiquitin promoter-based vectors for high-level expression of selectable and/or screenable marker genes in monocotyledonous plants. Transgenic Res 5:213-218, and is publicly available from the Institute of Genetics and Developmental Biology, Chinese Academy of Sciences; and the modified pACH25 vector is obtained by inserting an Ubiquitin promoter into the pACH25 vector between PstI enzyme cleavage sites.

“Longchun 23” wheat is disclosed in Junxiu Yuan, Wenxiong Yang. A new variety of high-quality spring wheat of high yield and wide adaptability—“Longchun 23”[J], Journal of Triticeae Crops, 2009, 29(4): 740″, and is publicly available from the Institute of Genetics and Developmental Biology, Chinese Academy of Sciences.

In the Examples, the high-nitrogen nutrient solution and low-nitrogen nutrient solution with nitrogen element concentrations of 2 mM and 0.2 mM, respectively were formulated as shown in Table 1.

TABLE 1
Formulations of high- and low-nitrogen nutrient solutions

| | High-nitrogen | Low-nitrogen |
|---|---|---|
| | nutrient | nutrient |
| | solution | solution |
| | (mM) | (mM) |
| KH₂PO₄ | 0.2 | 0.2 |
| Ca(NO₃)₂•4H₂O | 1 | 0.1 |
| MgSO₄•7H₂O | 1.0 | 1.0 |
| KCl | 1.5 | 1.5 |
| CaCl₂ | 1.5 | 2.45 |
| H₃BO₃ | 0.001 | 0.001 |
| (NH₄)₆Mo₇O₂₄•4H₂O | 0.00005 | 0.00005 |
| CuSO₄•5H₂O | 0.0005 | 0.0005 |
| ZnSO₄•7H₂O | 0.001 | 0.001 |
| MnSO₄•2H₂O | 0.001 | 0.001 |
| EDTA-FeNa | 0.1 | 0.1 |

To each of above systems, an aqueous solution of 0.04 g/100 ml MES (2-(4-morpholinyl) ethyl sulfonate) was added to adjust pH to 5. Both of the high-nitrogen nutrient solution and the low-nitrogen nutrient solution were composed of water in addition to above components.

### Example 1. Construction of TaNAC2 Gene Transformed Wheat

I. Preparation of TaNAC2 Gene Transformed Plant

(I) Obtaining TaNAC2 Gene

1. Total RNA was extracted from “Chinese Spring” wheat, and was subjected to reverse transcription, to obtain genomic cDNA thereof.

2. PCT amplification was conducted, using the cDNA obtained in step 1 as a template, with following primers:

upstream primer:
(SEQ ID No. 1)
5′- GGATCCATGGGGATGCCGGCCGTG -3′
(underlined sequence represents BamHI enzyme
recognition site)
downstream primer:
(SEQ ID No. 2)
5′- GGTACCGAACGGGGCCGGCATGC -3′
(underlined sequence represents KpnI enzyme
recognition site)

PCR system (40 μl): 4 μl of template cDNA, 1 μl of KOD plus DNA polymerase, 4 μl of 10×PCR buffer for KOD plus, 4 μl of dNTPs (2 mM each), 2 μl of 25 mM MgSO₄, upstream and downstream primers each 20 mM, supplemented with double distilled water to 40 μl reaction system.

PCR reaction procedure: 98° C. for 2 min; 35 cycles of 98° C. for 30 sec, 58° C. for 30 sec, and 68° C. for 45 sec.

PCR amplification product is represented by SEQ ID No.3, the nucleotide sequence of TaNAC2 gene is represented by 7^{th} to 993^{rd} nucleotide from 5′ end of SEQ ID No.3, and the amino acid sequence of TaNAC2 protein is represented by SEQ ID No.4.

(II) Construction of TaNAC2 Gene Cloned Vector

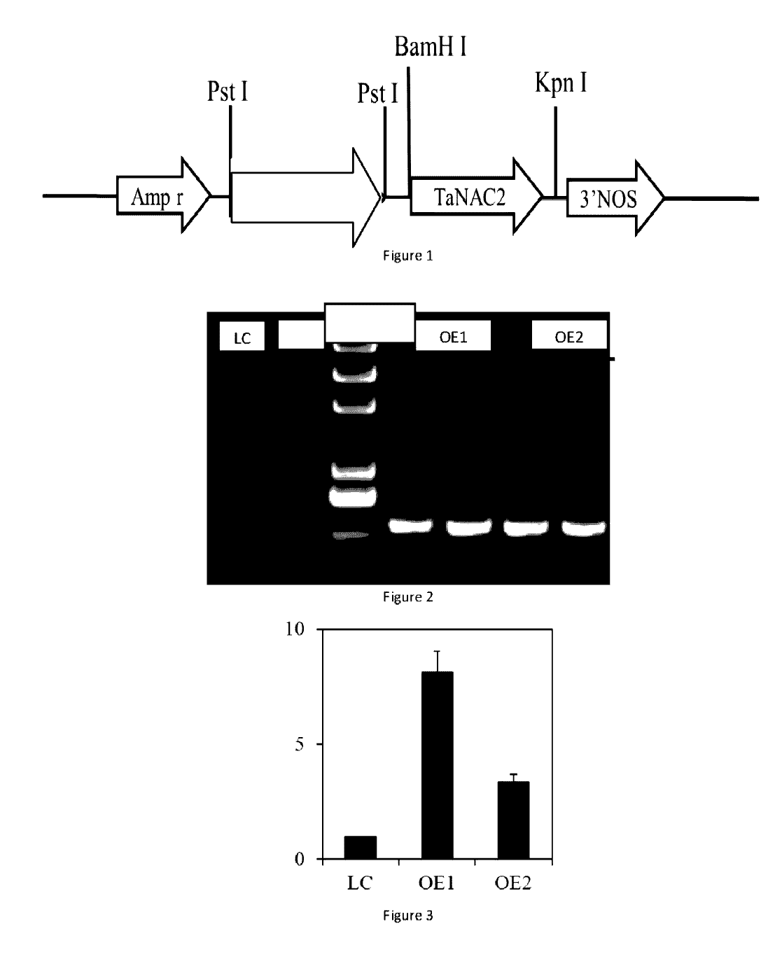

The DNA molecule represented by SEQ ID No.3 was double digested with BamHI and KpnI, to obtain a gene fragment; a modified pACH25 vector was double digested with BamHI and KpnI, to obtain a larger vector fragment, the gene fragment was ligated to the larger vector fragment, to obtain a recombinant plasmid, designated as pACH25/TaNAC2; the recombinant plasmid was sent for sequencing, and the result showed it was correct. The TaNAC2 gene in pACH25/TaNAC2 is promoted by Ubiquitin promoter, as shown in FIG. 1.

(III) Obtaining Transgenic Wheat

The pACH25-TaNAC2 was transformed into the wheat “Longchun 23” by means of a gene gun, to obtain T0 TaNAC2 gene transformed wheat. Genomic DNA was extracted from a leaflet of T0 TaNAC2 gene transformed wheat, and used as a template to conduct PCR amplification with a forward primer and a reverse primer, to obtain a fragment of about 500 bp, i.e., positive T0 TaNAC2 gene transformed wheat.

(SEQ ID No. 5)
pF: 5′- TTAGCCCTGCCTTCATACGC -3′
(SEQ ID No. 6)
pR: 5′- CAGTCGGTCTTGACCCCCTTA -3′

Above positively identified T0 TaNAC2 transformed wheat was cultivated to T3, with T1-T3 plants identified using a method as the identification of T0, followed by screening a homozygous line of T3 TaNAC2 gene transformed plants (i.e., the line where all the T2 seeds allow for the next generation of positive TaNAC2 gene transformed plants as identified by PCR is identified as a homozygous line), and the seeds thereof were harvested, and all of subsequent experiments used the T3 TaNAC2 gene transformed homozygous line (abbreviated as “a T3 TaNAC2 transformed wheat line” hereinafter).

The wheat “Longchun 23” was also transformed with vector pACH25 using above method, to obtain blank vector transformed wheat.

II. Detection and Phenotype Analysis of Transgenic Plant

(I) Detection at DNA Level

DNA was extracted from a leaflet of T3 TaNAC2 gene transformed wheat, blank vector transformed wheat, and wild-type wheat “Longchun 23”, and used as a template to conduct PCR amplification with primers of pF and pR, respectively, and water was used as blank control.

PCR reaction system (20 μl):

| | |
|---|---|
| DNA template (about 20 ng/μl) | 2 μl |
| pF (10 μM) | 0.5 μl |
| pR (10 μM) | 0.5 μl |
| 10x PCR amplification buffer | 2 μl |
| dNTP Mixture | 1 μl |
| TaqDNA polymerase | 0.2 μl |
| ddH₂O | 13.8 μl |

PCR reaction procedure: 94° C. for 3 min; 40 cycles of 94° C. for 30 s, 60° C. for 30 s, and 72° C. for 40 s; and 72° C. for 5 min.
The target PCR amplification band of TaNAC2 gene is of about 500 bp, and the results are shown in FIG. 2.

In FIG. 2, LC: wild-type wheat “Longchun 23”; OE1 and OE2 represent two T3 TaNAC2 transformed wheat lines.

FIG. 2 shows that wild-type wheat “Longchun 23” did not have the target band, and the two T3 TaNAC2 transformed wheat lines OE1 and OE2 were preliminarily identified as positive TaNAC2 gene transformed wheat.

The blank vector transformed wheat had the same experimental results as those of the wild-type wheat “Longchun 23”.

(II) Detection at RNA Level

1. Total RNA was extracted from a leaflet of T3 TaNAC2 gene transformed wheat, blank vector transformed wheat and wild-type wheat “Longchun 23”, and reversely transcribed to cDNA, respectively.

2. The cDNAs obtained in step 1 were respectively used as a template to conduct RT-PCR with primers of TaNAC2 RT pF and TaNAC2 RT pR to amplify TaNAC2 gene, and meanwhile to conduct RT-PCR with primers of Tublin pF and Tublin pR to amplify an internal reference gene, Tublin.

The primers are as below:

upstream primer TaNAC2 RT pF:
(SEQ ID No. 7)
5′- CTGGGTGCTCTGCCGGCTCTAC-3′
downstream primer TaNAC2 RT pR:
(SEQ ID No. 8)
5′- CTCCGCCTTGGGCTCCATCATC-3′
upstream primer Tublin pF:
(SEQ ID No. 9)
5′- ACCGCCAGCTCTTCCACCCT -3′
downstream primer Tublin pR:
(SEQ ID No. 10)
5′- TCACTGGGGCATAGGAGGAA -3′

PCR System:

| | |
|---|---|
| DNA template (about 20 ng/μl) | 2 μl |
| upstream primer (10 μM) | 0.4 μl |
| downstream primer (10 μM) | 0.4 μl |
| 2x mixture | 10 μl |
| (light Cycler SYBR Green I master, purchased from Roche) | |
| ddH₂O | 7.2 μl |
| total volume | 20 μl |

PCR procedure: 94° C. for 5 min; 40 cycles of 94° C. for 10 s, 60° C. for 20 s, 72° C. for 15 s.

Quantitative analysis: Roche LightCycler 480 II realtime PCR machine was used for analyzing CT value. Taking Tublin as an internal reference and the relative expression amount of TaNAC2 gene in the wild-type wheat “Longchun 23” as 1, the expression of TaNAC2 gene in the T3 TaNAC2 transformed wheat and blank vector transformed wheat were relatively quantified with 2^{-ΔΔct}.

The detection results of the TaNAC2 gene in the two T3 TaNAC2 transformed wheat lines of OE1 and OE2 are shown in FIG. 3.

In FIG. 3, LC represents wild-type wheat “Longchun 23”.

FIG. 3 shows that, as compared with wild-type wheat “Longchun 23”, the expression amounts of TaNAC2 gene in the two T3 TaNAC2 transformed wheat lines of OE1 and OE2 were increased by 8.13 folds and 3.38 folds, respectively.

The experimental result of the blank vector transformed wheat was the same as that of the wild-type wheat “Longchun 23”.

From the DNA level detection in step (I) and the RNA level detection in step (II), it was confirmed that the two T3 TaNAC2 transformed wheat lines of OE1 and OE2 were successfully constructed.

### Example 2. Phenotype Identification of TaNAC2 Gene Transformed Wheat

I. Identification of root phenotype was performed in the condition of water cultivation, which particularly comprises steps of:

Germinating the harvested seeds of the T3 TaNAC2 transformed wheat (OE1 and OE2) and the wild-type wheat “Longchun 23” in an incubator at 23° C. (incubated with tap water) for 7 days, removing embryos therefrom, and transferring the resultants to a high-nitrogen nutrient solution or a low-nitrogen nutrient solution for cultivation.

II. After 14 days of the cultivation of the plants from step I, the length of main roots (the length of longest root) were measured with a ruler, and the number of lateral roots (lateral branch number) were analyzed with a WinRHIZO root scanning system.

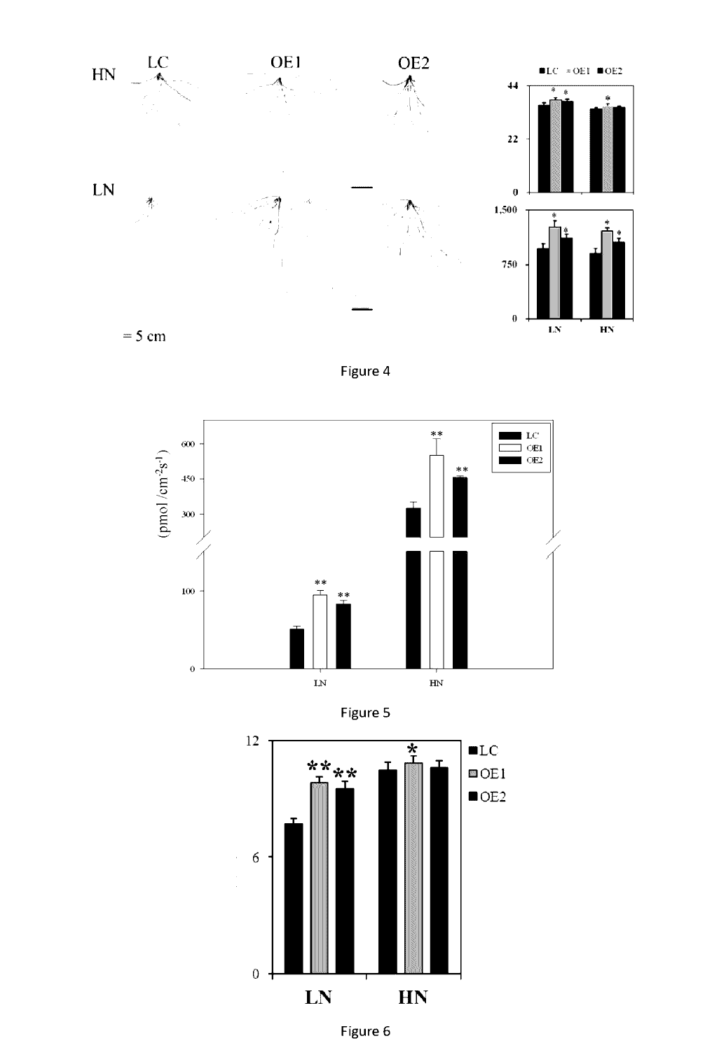

The results are shown in FIG. 4. In FIG. 4, LC represents the wild-type wheat “Longchun 23”, LN represents the cultivation results in the low-nitrogen nutrient solution, and HN represents the cultivation results in the high-nitrogen nutrient solution.

FIG. 4 shows that in the condition of low-nitrogen cultivation, the wild-type wheat “Longchun 23” had an average lateral branch number of 972.33, an average length of the longest root of 36.03 cm; OE1 had an average lateral branch number of 1271.6, and an average length of the longest root of 38.15 cm; OE2 had an average lateral branch number of 1112.4 t, and an average length of the longest root of 37.68 cm. In the condition of high-nitrogen cultivation, the wild-type wheat “Longchun 23” had an average lateral branch number of 910.4, and an average length of the longest root of 34.4 cm; OE1 had an average lateral branch number of 1218.33, and an average length of the longest root of 35.7 cm; OE2 had an average lateral branch number of 1058.67, and an average length of the longest root of 35.18 cm.

The results suggest that the TaNAC2 transformed wheat had an significant increase of lateral root number and an increase of the length of the longest root relative to that of the wild-type wheat “Longchun 23”, regardless of in the condition of high-nitrogen cultivation or in the condition of low-nitrogen cultivation.

III. After 7 days of the cultivation of the plants from step I, nitrate uptake rate was measured using a BIO-IM non-invasive measuring system (YoungerUSA, LLC) with related parameters selected according to Luo J, Qin J, He F, et al. Net fluxes of ammonium and nitrate in association with H+ fluxes in fine roots of Populus popularis[J]. Planta, 2013, 237(4): 919-931.

The results are shown in FIG. 5. In FIG. 5, LC represents the wild-type wheat “Longchun 23”, LN represents the cultivation results in the low-nitrogen nutrient solution, HN represents the cultivation results in the high-nitrogen nutrient solution.

FIG. 5 shows that, in the low-nitrogen condition, the nitrate uptake rate was 51.17 pmol/cm⁻²·s⁻¹ for wild-type wheat “Longchun 23”, 94.63 pmol/cm⁻²·s⁻¹ for OE1, and 83.46 pmol/cm²·s⁻¹ for OE2; in the high-nitrogen condition, the nitrate uptake rate was 325.21 pmol/cm⁻²·s⁻¹ for wild-type wheat “Longchun 23”, 551.63 pmol/cm⁻²·s⁻¹ for OE1, and 455.64 pmol/cm⁻²·s⁻¹ for OE2.

FIG. 5 indicates that, under the condition of water cultivation, the nitrate uptake rate of the root system of the TaNAC2 transformed wheat lines was significantly higher than that of the wild-type wheat “Longchun 23”, regardless of high-nitrogen cultivated or low-nitrogen cultivated.

IV. Phenotype identification of tiller number and aboveground dry weight was performed in the condition of pot cultivation, particularly comprising steps of:

sowing seeds uniformly germinated after kept in dark at 23° C. for 2 days, selected from the harvested seeds of the T3 TaNAC2 transformed wheat (OE1 [symbol] OE2) and the wild-type wheat “Longchun 23”, in pots each containing 3 kg earth at the spring sowing time of wheat (with 100 mg N/kg earth of a high-nitrogen fertilizer, and 0 mg N/kg earth of a low-nitrogen fertilizer applied); after 4-week growth at an open area under natural light conditions, counting tiller number, then harvesting aboveground parts, and drying these parts at 105° C. for 15 min and at 70° C. until having constant weight, then weighting the aboveground dry weight.

The statistical results of the tiller number are shown in FIG. 6. In FIG. 6, LC represents the results of the wild-type wheat “Longchun 23”, LN represents the results of low-nitrogen cultivation, and HN represents the results of the high-nitrogen cultivation.

FIG. 6 shows that, in the condition of low-nitrogen pot cultivation, wild-type wheat “Longchun 23” had an average tiller number of 7.71, OE1 had an average tiller number of 9.83, and OE2 had an average tiller number of 9.5; in the condition of high-nitrogen pot cultivation, wild-type wheat “Longchun 23” had an average tiller number of 10.47, OE1 had an average tiller number of 10.83, and OE2 had an average tiller number of 10.58.

FIG. 6 indicates that, in a low-nitrogen condition, the TaNAC2 transformed plants had a tiller number at seeding stage significantly higher than that of the wild-type wheat “Longchun 23”.

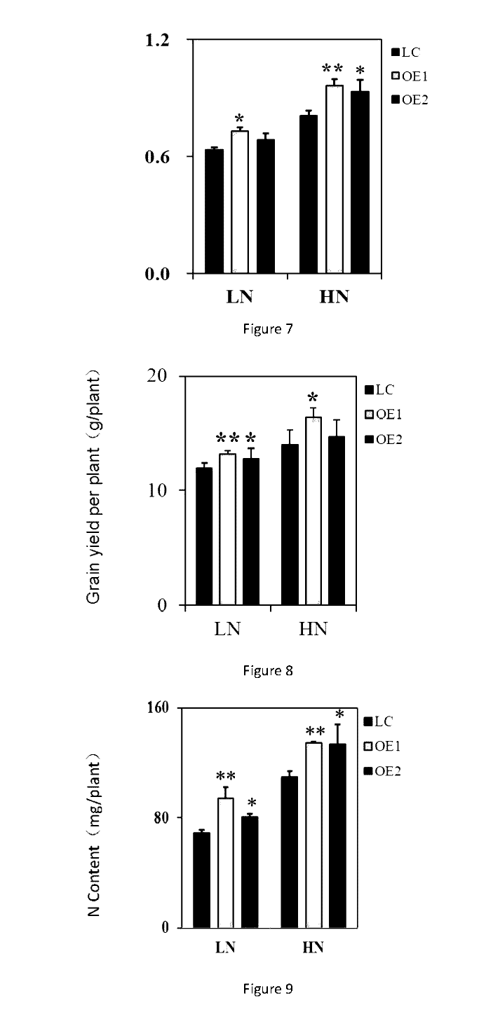

The results of the aboveground dry weight are shown in FIG. 7. In FIG. 7, LC represents the results of wild-type wheat “Longchun 23”, LN represents the results of low-nitrogen cultivation, and HN represents the results of the high-nitrogen cultivation.

FIG. 7 shows that, in the condition of low-nitrogen pot cultivation, wild-type wheat “Longchun 23” had an average aboveground dry weight of 0.64 g, OE1 had an average aboveground dry weight of 0.73 g, and OE2 had an average aboveground dry weight of 0.69 g; in the condition of high-nitrogen pot cultivation, wild-type wheat “Longchun 23” had an average aboveground dry weight of 0.81 g, OE1 had an average aboveground dry weight of 0.96 g, OE2 had an average aboveground dry weight of 0.93 g.

FIG. 7 indicates that the TaNAC2 transformed plants had better growth and significantly higher aboveground biomass than that of wild-type wheat “Longchun 23” at seeding stag, in both of high- and low-nitrogen conditions.

V. Grain yield per plant was measured in field conditions at Changping District, Beijing, China, particularly comprising steps of:

sowing 3 rows of the resultant seeds of T3 TaNAC2 transformed wheat (OE1 and OE2) and wild-type wheat “Longchun 23” respectively in each of areas, in quadruplicate, with a row length of 1.5 m and a plant spacing of 5 cm, to measure the grain yield per plant, wherein the cultivation was separately performed in the low-nitrogen condition with no nitrogen fertilizer applied and in the high-nitrogen condition with 15 kg N/mu of nitrogen fertilizer applied.

The results are shown in FIG. 8. In FIG. 8, LC represents the results of the wild-type wheat “Longchun 23”, LN represents the results of the low-nitrogen cultivation, and HN represents the results of the high-nitrogen cultivation.

FIG. 8 shows that, in the condition of low-nitrogen field cultivation, wild-type wheat “Longchun 23” had an average grain yield per plant of 12.0 g, OE1 had an average grain yield per plant of 13.2 g, and OE2 had an average grain yield per plant of 12.8 g; in the condition of high-nitrogen field cultivation, LC had an average grain yield per plant of 14.0 g, OE1 had an average grain yield per plant of 16.4 g, and OE2 had an average grain yield per plant of 14.7 g.

The results indicate that, the grain yield per plant of the TaNAC2 transformed wheat was significantly higher than that of the wild-type wheat “Longchun 23”, in both of high- and low-nitrogen conditions.

VI. Determination of Nitrogen Content

(I) Determination of Nitrogen Content in Plant

The harvested seeds of T3 TaNAC2 transformed wheat (OE1 and OE2) and wild-type wheat “Longchun 23” were germinated an incubator at 23° C. (incubated with tap water). After 7 days, embryos were removed, and the resultants were transferred to a high-nitrogen nutrient solution or a low-nitrogen nutrient solution for cultivation. After a 14-day cultivation, samples of the seeding stage T3 TaNAC2 transformed wheat (OE1 and OE2) and wild-type wheat “Longchun 23” were weighted with a balance of 1/10000 scale, ground with a cyclone mill A 100 mL of Kelvin bottle or a “cooking” tube was charged with 0.3˜0.5 g of a sample as above at bottom, and added 5 mL of concentrated H₂SO₄, shaken until homogenous (preferably left to stand overnight), and heated mildly in an electric furnace or a “cooking” furnace, then at an elevated temperature when H₂SO₄ fumed. When the solution appeared uniformly dark brown, the bottle or tube was removed from the furnace, added with 10 droplets of 30% H₂O₂ when slightly cooled, and then heated again to slight boiling, cooking for about 7˜10 min. When it was slightly cooled, another part of 30% H₂O₂ was added, and cooked again. The same was repeated several times, with the added amounts of H₂O₂ gradurally decreased. When the solution was cooked to appear colorless or clear, heating was continued for about 10 min. Then, residual H₂O₂ was removed, and the residues were cooled to room temperature and supplemented to a constant volume for nitrogen content determination. The nitrogen content determination was performed by means of indophenol blue colorimetry of Novozamsky et al. (Novozamsky I, Eck R van, Schouwenburg J C van, et al. Total nitrogen determination in plant material by means of the indophenol-blue method [J]. Netherlands Journal of Agricultural Science, 1974, 22(1): 3-5).

The results are shown in FIG. 9. In FIG. 9, LC represents the results of the wild-type wheat “Longchun 23”, LN represents the results of the low-nitrogen cultivation, and HN represents the results of the high-nitrogen cultivation.

FIG. 9 shows that, in the condition of low-nitrogen water cultivation, an average of total N content per plant was 68.77 mg for wild-type wheat “Longchun 23”, 94.21 mg for OE1, and 80.42 mg for OE2; in the condition of high-nitrogen water cultivation, an average of total N content per plant was 109.63 mg for wild-type wheat “Longchun 23”, 134.39 mg for OE1, and 133.67 mg for OE2.

The results indicate that TaNAC2 transformed wheat had a total nitrogen content at seeding stage higher than that of wild-type wheat “Longchun 23”.

(II) Determination of Grain Nitrogen Concentration

Grains were harvested from T3 TaNAC2 transformed wheat (OE1 and OE2) and wild-type wheat “Longchun 23” obtained in step V after the entire growth period of 108 days, for nitrogen content determination by the same method as in step (I).

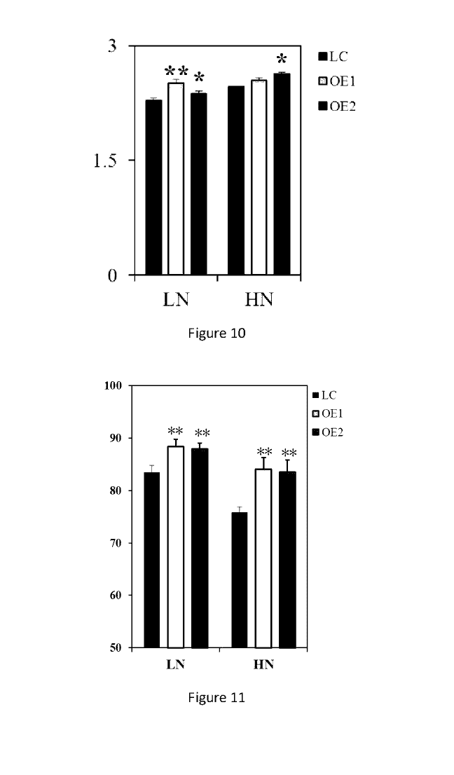

The results are shown in FIG. 10. In FIG. 10, LC represents the results of the wild-type wheat “Longchun 23”, LN represents the results of the low-nitrogen cultivation, and HN represents the results of the high-nitrogen cultivation.

FIG. 10 shows that, in the condition of low-nitrogen field cultivation, the grain nitrogen concentration was 2.28% for wild-type wheat “Longchun 23”, 2.51% for OE1, and 2.37% for OE2; in the condition of high-nitrogen field cultivation, the grain nitrogen concentration was 2.47% for wild-type wheat “Longchun 23”, 2.54% for OE1, and 2.63% for OE2.

The results indicate that the nitrogen concentration in the grains of the TaNAC2 transformed wheat was significantly higher than that of the wild-type wheat “Longchun 23”.

(III) Determination of Nitrogen Element Harvest Index

Nitrogen element harvest index is an important physiological index to measure the utilization efficiency of nitrogen element by a plant, wherein

Nitrogen element harvest index=nitrogen content in grains/total nitrogen content in aboveground parts,

A high nitrogen element harvest index indicates a high utilization efficiency of nitrogen element, which is beneficial to saving fertilizers, improving economic benefit, and reducing environmental pollution.

Grains and aboveground parts were harvested from T3 TaNAC2 transformed wheat (OE1 and OE2) and wild-type wheat “Longchun 23” obtained in step V after the entire growth period of 108 days for nitrogen content determination by a method as in step (I).

The results are shown in FIG. 11. In FIG. 11, LC represents the results of the wild-type wheat “Longchun 23”, LN represents the results of the low-nitrogen cultivation, and HN represents the results of the high-nitrogen cultivation.

FIG. 11 shows that, in the condition of low-nitrogen field cultivation, the utilization efficiency of nitrogen element was 83.49% fir wild-type wheat “Longchun 23”, 88.46% for OE1, and 87.97% for OE2; in the condition of high-nitrogen field cultivation, the utilization efficiency of nitrogen element was 75.91% for wild-type wheat “Longchun 23”, 84.09% for OE1, and 83.60% for OE2.

The results indicate that the TaNAC2 transformed wheat had a nitrogen element harvest index significantly higher than that of the wild-type wheat “Longchun 23”.

### INDUSTRIAL APPLICATION

The disclosure of the present invention that the TaNAC2 gene, as a nitrate nitrogen responsive regulatory factor, which can regulate the expression of a series of genes in nitrogen element uptake pathways of wheat, greatly promotes the mechanism study for illuminating the metabolism and utilization of the nitrogen element in a plant. It is possible to improve the assimilation efficiency of nitrogen element by increasing the expression of TaNAC2 through genetic engineering, thereby realizing the purposes of reducing fertilizer application and increasing yield and the like.

1. A method for improving uptake, transport and/or assimilation efficiency of nitrogen element in a plant, comprising:
introducing a coding gene of a protein represented by SEQ ID No.4 into a primary plant, to obtain a transgenic plant,
wherein the transgenic plant, compared with the primary plant, has improved uptake, transport and/or assimilation efficiencies of the nitrogen element.

2. The method according to claim 1, wherein
the coding gene is introduced via a recombinant vector which is obtained by replacing GUS gene sequence with the coding gene in a modified pACH25;
the modified pACH25 is obtained by inserting an Ubiquitin promoter into a pACH25 vector between PstI enzyme cleavage sites;
the method for introducing a coding gene of a protein represented by SEQ ID No.4 into a primary plant is a transformation method mediated by a gene gun; and
the coding gene has a nucleotide sequence represented by 7^{th} to 993^{rd} nucleotide from 5′ end of SEQ ID No.3.

3. The method according to claim 1, wherein the plant is wheat.

4. (canceled)

5. The method according to claim 1, wherein the transgenic plant, compared with the primary plant, has enhanced
growth, development and/or grain production.

6. The method according to claim 1, wherein the transgenic plant, compared with the primary plant, has enhanced
root growth.

7. The method according to claim 1, wherein the transgenic plant, compared with the primary plant, has increases in
main root length and/or lateral root number.

8. The method according to claim 1, wherein the transfenic plant, compared with the primary plant has improved
nitrate uptake rate.

9. The method according to claim 1, wherein the transgenic plant, compared with the primary plant, has improvement in one or more of the following:
tiller number or aboveground dry weight of a plant, grain yield per plant of a plant, nitrogen content in the body of a plant, or nitrogen content in the grains of a plant.

10. (canceled)

11. The method according to claim 2, wherein said replacing GUS gene sequence with the coding gene in a modified pACH25 comprises:
inserting a DNA molecule represented by SEQ ID No.3 into the modified pACH25 between BamHI and KpnI enzyme cleavage sites.
